(19) United States
(12) Patent Application Publication
(10) Pub. No.: US 2004/0097784 A1
(43) Pub. Date: May 20, 2004

(54) Intraluminal inflatable counter-pulsation heart assist device

(75) Inventors: William Suttle Peters, Auckland (NZ); Hans Hansforth Henrichsen, Wollongong (AU); Scott Hugh Miller, Manly (AU); Rodney Gordon Parkin, Crows Nest (AU)

Correspondence Address: ORRICK, HERRINGTON & SUTCLIFFE, LLP, 4 PARK PLAZA, SUITE 1600, IRVINE, CA, 92614-2558, US

(73) Assignee: SUNSHINE HEART COMPANY PTY LTD.

(21) Appl. No.: 10/634,642

(22) Filed: Aug. 4, 2003

(30) Foreign Application Priority Data
Nov. 15, 2002 (AU) 2002952730

(51) Int. Cl. A61N 1/362

(52) U.S. Cl. 600/18; 600/16

(57) ABSTRACT

Apparatus and method for performing a heart assist function by use of intraluminal inflatable counter-pulsation balloon or chamber. The apparatus includes means to hold the balloon or chamber in place against an inner wall of a blood vessel. The balloon or chamber is connected to a source of pressure which expands and contracts the balloon in counter-pulsation with the heart.

### RELATED INFORMATION

This application claims priority of provisional Australian application number 2002952730 filed on Nov. 15, 2002.

### FIELD OF THE INVENTION

The present invention relates generally to an intraluminal inflatable counter-pulsation heart assist device and more particularly to a counter-pulsation stent or stent graft.

### BACKGROUND OF THE INVENTION

It is known to provide counter-pulsation through intra-aortic balloons (IAB) which float freely in the blood stream and through an aortic patch which must be sewn into the wall of a vessel. In the case of the IAB the balloon is inserted percutaneously into a peripheral artery and moved endovascularly into the aorta. The balloon is counter pulsated by a console external to the body and patient mobility is extremely limited. The aortic patch is inserted into the body through a thoracotomy and requires both cardio-pulmonary by-pass and an incision in the aorta. The aortic patch is also driven from a device outside the patients body but this device does allow some patient mobility.

It would be desirable to provide a blood contacting counter-pulsation heart assist device that is placed within the aorta, or other artery, intraluminally to avoid the necessity for surgery while allowing patient mobility. Such a device would also desirably minimise or at least reduce the area of the device in contact with the blood, when compared to existing blood contacting devices.

### SUMMARY OF THE INVENTION

In a first aspect, the present invention provides a heart assist device including an intraluminal inflatable counter-pulsation balloon, or chamber, adapted to be held in place against a wall of the aorta, or other suitable arterial vessel, of a patient, the inflatable balloon or chamber being connectable to a fluid pressure source adapted to cause the inflatable balloon or chamber to be expanded and contracted in counter-pulsation with the heart of a patient into whom the balloon or chamber has been placed.

The chamber or balloon is preferably part of a stent or stent-graft comprising an expandable frame, the counter-pulsation balloon or chamber being attached to the inside wall of the frame. The frame of the stent or stent-graft may be self expanding, expandable by a conventional balloon catheter or expandable by hyper-inflation of the chamber itself. If it is self-expanding it may be formed of a spring material or of a shape memory alloy such as Nitinol.

The stent or stent-graft, including the inflatable counter-pulsation balloon or chamber, is preferably adapted to be packaged into a catheter delivery structure which can be introduced into a suitable artery, typically the ascending or descending aorta. The catheter may be introduced into the vasculature upwardly, such as from the femoral artery, or downwardly, such as from the carotid or sub-clavian artery.

The frame of the stent or stent-graft is preferably formed of wires. The frame may be covered with a fabric, such as Dacron, or have a coating such as of Teflon, around its periphery on either the outside or the inside of the frame. Preferably the wires of the stent or stent-graft will be bare adjacent any vessels branching off from a vessel into which the stent or stent-graft is placed.

The inflatable counter-pulsation balloon or chamber may extend around the full circumference of the lumen of the frame of the stent or stent-graft or, and preferably, it may extend around only a part of that circumference. In the latter case the part of the stent or stent-graft over which the balloon of chamber does not extend may be formed as a bare stent so that any branch vessels diverging from the artery in which the stent is positioned will not be occluded.

The stent is preferably placed percutaneously and then connected to a drive mechanism by forming an aperture in the wall of the aorta or other artery and connecting a fluid conducting tube from the drive mechanism to the balloon or chamber. More preferably, the fluid conducting tube can be a gas carrying tube which exits the body percutaneously and is coupled to a suitable pneumatic driver. In this case the stent or stent-graft can be placed intraluminally and the gas line connected to a port on the stent or stent-graft surgically, preferably thoracoscopically.

In an alternative embodiment, the stent may be placed intraluminally and then connected to an hydraulic driver placed in the chest through a sternotomy or thoracotomy. A liquid drive line in this case being connected through an aortotomy to a port on the stent which is in communication with the interior of the balloon or chamber.

In another embodiment, the stent or stent-graft is placed through a stab wound in the aorta, preferably the ascending aorta or the thoracic descending aorta. In this case the aorta may be approached through a sternotomy. A purse string is then placed around a site for a stab incision and drawn tight around a catheter used for placement of the stent in the aorta. In this embodiment the pump may be attached to the balloon within the stent or stent-graft prior to the placement of the stent or stent-graft in the aorta.

In a second aspect, the present invention provides a method of assisting the functioning of a heart of a patient, the method including the steps of:

holding a heart assist device, that includes an intraluminal inflatable counter-pulsation balloon, or chamber, in place against a wall of an arterial vessel of the patient;

connecting the inflatable balloon or chamber to a fluid pressure source adapted to cause the inflatable balloon or chamber to be expanded and contracted in counter-pulsation with the heart of a patient into whom the balloon or chamber has been placed.

The chamber or balloon is preferably part of a stent or stent-graft comprising an expandable frame and the method preferably includes the step of attaching the counter-pulsation balloon or chamber to the inside wall of the frame.

The method preferably also includes the step of packaging the stent or stent-graft, including the inflatable counter-pulsation balloon or chamber, into a catheter delivery structure and introducing the structure into a suitable artery, typically the ascending or descending aorta. The catheter may be introduced into the vasculature upwardly, such as from the femoral artery, or downwardly, such as from the carotid or subclavian artery.

The method preferably also includes the step of placing the stent or stent-graft intraluminally and then connecting it to a drive mechanism by forming an aperture in the wall of the aorta or other artery and connecting a fluid conducting tube from the drive mechanism to the balloon or chamber. More preferably, the fluid conducting tube can be a gas carrying tube which exits the body percutaneously and is coupled to a suitable pneumatic driver. In this case the stent or stent-graft can be placed intraluminally and the gas line connected to a port on the stent thoracoscopically.

In an alternative embodiment, the stent or stent-graft may be placed intraluminally and then connected to an hydraulic driver placed in the right chest through a sternotomy. A liquid drive line in this case being connected through an aortotomy to a port on the stent or stent-graft which is in communication with the interior of the balloon or chamber.

In another embodiment, the stent is placed through a stab wound in the aorta, preferably the ascending aorta. In this case the aorta is approached through a sternotomy. A purse string is then placed around a site for a stab incision and drawn tight around a catheter used for placement of the stent in the aorta. In this embodiment the pump may be attached to the balloon within the stent or stent-graft prior to the placement of the stent in the aorta.

### BRIEF DESCRIPTION OF THE DRAWINGS

Preferred embodiments of the invention will now be described, by way of examples only, with reference to the accompanying drawings in which:

FIG. 1 is a partially cutaway view of the aorta of a patient into the ascending aspect of which an embodiment of a device according to the invention has been placed intraluminally;

FIG. 2 is a cross sectional view of the aorta and device of FIG. 1 along line 11-11;

FIG. 3 is a partly cut away ventral view of the chest of a patient with an embodiment of a device according to the present invention placed in the thoracic descending aorta;

FIG. 4 is a longitudinal dorso-ventral sectional view through the aorta and device of the patient shown in FIG. 3; and

FIG. 5 is a partially cutaway view of the aorta of a patient into the ascending aspect of which an embodiment of a device according to the invention has been placed by sternotomy, with the balloon in an inflated condition.

### DETAILED DESCRIPTION OF THE PREFERRED EMBODIMENTS

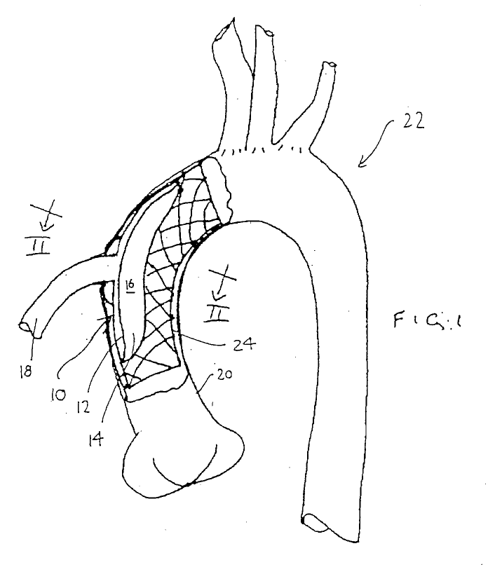

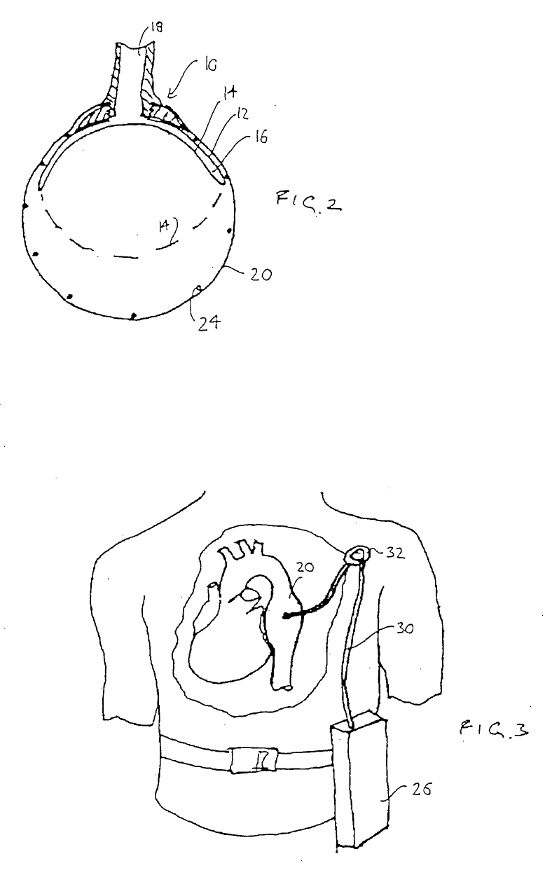

Referring firstly to FIGS. 1 and 2 there is shown a first embodiment of an intravascular heart assist device 10 according to the invention. The device 10 includes an inelastic, preferably plastic, shell 12 and a membrane 14, sealingly attached to periphery of the shell 12. The membrane 14 defines an inflatable space 16 between it and the interior of the shell 12. The shell 12 also has an inlet/outlet port 18 which is adapted for connection to a motive means (not shown) that can periodically introduce, and withdraw, a fluid (eg., a gas such as helium or a liquid such as a saline solution or an oil) to and from the space 16 (to expand and retract the membrane 14 respectively) in counter-pulsation with the patient's heart rhythm.

Turning to FIG. 2, the solid line of the membrane 14 shows the membrane 14 after withdrawal of fluid from the space 16. The phantom line shows the membrane 14 after introduction of fluid into the space 16 and expansion of the membrane 14. A general explanation of heart assistance utilising aortic counter-pulsation can be found in the Applicant's international PCT patent application no. PCT/AU00/00654, now entitled “Heart Assist Devices, Systems and Methods”, which is incorporated herein by cross-reference.

Returning to FIG. 1, the device 10 is shown positioned in the ascending aspect 20 of a patient's aorta 22. FIG. 1 also shows that the device 10 is attached to the inside wall of a stent-graft formed form an expandable frame 24. The frame 24 is covered with a fabric, such as Dacron, or other external coating such as Teflon around its periphery on either the outside or the inside of the frame 24. The wires of the frame 24 are bare adjacent any vessels branching off from the aorta 20 so that such vessels are not occluded. It should also be noted that the shell 12 extends around only a part of the circumference of the frame 24 and any part of the frame 24 over which the shell 12 or membrane 14 does not extend are formed bare, again to avoid occlusion of any branching vessels diverging from the aorta 20.

The frame 24 may be self expanding and formed of a spring material or of a shape memory alloy such as Nitinol. Alternatively, the frame 24 may be expandable under the influences of the expansion of the membrane 14, as will be describe [sic] in more detail below. In another alternative a conventional balloon catheter could be used to expand the frame 24.

As best seen in FIG. 2, under normal operating conditions, the membrane is expanded (see phantom line) to a diameter less than that of the expanded frame 24. Accordingly, to expand the frame 24 with the membrane 14, the membrane 14 is initially expanded by a larger than normal amount and then a lesser amount of expansion is used in counter-pulsation with the patient's heart.

The frame 24, the shell 12 and the membrane 14 are packaged into a catheter delivery structure (not shown) which can be introduced into the aorta 20. Such structures are well known to persons skilled in the art and shall not be described in any more detail.

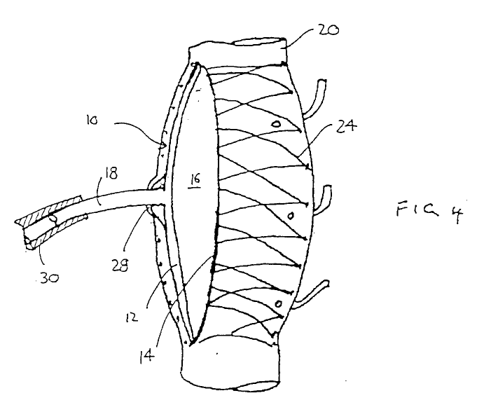

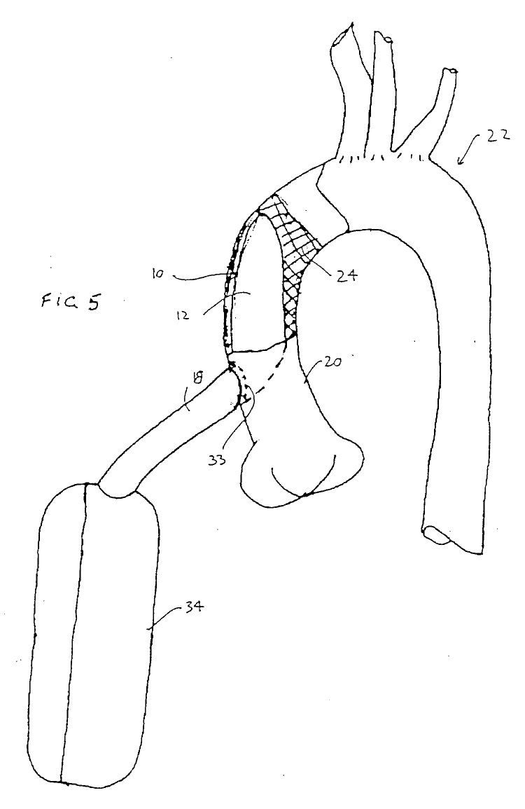

FIGS. 3 and 4 show a patient after the frame 24 has been placed intraluminally and then connected to a drive mechanism 26. This is done by forming an aperture 28 (see FIG. 4) in the wall of the aorta 20 and connecting a fluid connecting tube 30 from the drive mechanism 26 to the inlet/outlet port 18 of the device 10. More particularly, the fluid connecting tube 30 is a gas carrying tube which exists [sic] the body percutaneously at the opening 32 and the drive mechanism 26 is a suitable pneumatic driver.

FIG. 5 shows an alternative arrangement in which the frame 24 is positioned through a stab would in the aorta, preferably the ascending aorta. In this case the aorta is approached through a sternotomy. A purse string 33 is then placed around a site for a stab incision and drawn tight around the catheter used for placement of the frame in the aorta. In this embodiment, a driver 34 may be connected to the device prior to the placement of the device in the aorta. Suitable hydraulic drivers are disclosed in the Applicant's international PCT patent application no. PCT/AU02/00974 entitled “A Fluid Pressure Generating Means”, which is incorporated herein by cross reference and are capable of cyclically inflating and deflating the space 16. The driver 34 may be connected to one end of the shell 12, as is shown in FIG. 5, or intermediate its ends.

The embodiments of the invention described above possess a number of major advantages over prior art devices. Firstly, by positioning and holding the shell 12 of the device 10 against a wall of the aorta, the blood contacting surfaces of the device 10 are minimised as blood is not able to flow over the surfaces of the shell 12 adjacent the aorta wall. Secondly, where a patient has a vasculature that requires the expansion and/or support of a stent, and also a heart that requires the assistance of an inflatable counter-pulsation heart assist device, then both these objectives can be achieved with only a single surgical procedure, thus reducing the risk of surgical complications. Further, in some embodiments, the heart assist device itself can also be used to expand the stent. The devices may be placed with limited surgical intervention as compared with, for instance, the aortic patch. If there were plaque present in the aorta of a patient it may be possible to immobilise this plaque when placing the device bt trapping the plaque between the device and the aortic wall.

While the embodiments described above have been described in relation to positioning of an intraluminal inflatable counter-pulsation heart assist device within the ascending aorta, it would be appreciated by persons skilled in the art that such devices can be positioned in other parts of the aorta or in other arteries to assist in heart function.

The heart assist devices described above are suitable for short and long term treatment for heart failure and/or myocardial Ischemia.

It would also be appreciated by persons skilled in the art that numerous variations and/or modifications may be made to the invention as shown in the specific embodiments without departing from the spirit or scope of the invention as before described. The present embodiments are, therefore, to be considered in all respects as illustrative and not restrictive.

We claim:

1. A heart assist device comprising:
an intraluminal inflatable counter-pulsation balloon, or chamber; and
means to hold the balloon or chamber in place against an inner wall of an arterial vessel.

2. The heart assist device as claimed in claim 1, wherein the balloon or chamber forms part of a stent having an expandable frame.

3. The heart assist device as claimed in claim 2, wherein the counter-pulsation balloon or chamber is attached to an inner wall of the frame.

4. The heart assist device as claimed in claim 2, wherein the frame of the stent is self expanding.

5. The heart assist device as claimed in claim 4, wherein the frame is formed of a spring material.

6. The heart assist device as claimed in claim 2, wherein the frame is formed of a shape memory alloy.

7. The heart assist device as claimed in claim 2, wherein the frame is balloon or chamber expandable.

8. The heart assist device as claimed in claim 2, wherein the stent, including the balloon or chamber, is packaged into a catheter delivery structure which can be introduced into a suitable artery.

9. The heart assist device as claimed in claim 2, wherein the frame of the stent is formed of wires.

10. The heart assist device as claimed in claim 9, wherein the frame is covered with a fabric.

11. The heart assist device as claimed in claim 9, wherein the frame has a coating around its periphery on either the outside or the inside of the frame.

12. The heart assist device as claimed in claim 9, wherein the wires of the stent frame are bare adjacent any vessels branching off from a vessel into which the stent is placed.

13. The heart assist device as claimed in claim 2, wherein the frame defined a lumen and the balloon or chamber extends around the full circumference of the lumen of the frame.

14. The heart assist device as claimed in claim 2, wherein the frame defines a lumen and the balloon or chamber extends around a part of the circumference of the lumen of the frame.

15. The heart assist device as claimed in claim 14, wherein the part of the stent over which the balloon or chamber does not extend are formed as a bare stent so that any branch vessels diverging from the artery in which the stent is positioned will not be occluded.

16. The heart assist device as claimed in claim 1, further including a fluid conducting tube connected to the balloon or chamber.

17. The heart assist device as claimed in claim 2, further including a fluid pressure source connected to the balloon or chamber via the fluid conducting tube, the fluid pressure source adapted to cause the inflatable balloon or chamber to be expanded and contracted in counter-pulsation with the heart of a patient into whom the balloon or chamber has been placed.

18. The heart assist device as claimed in claim 17, wherein the stent is placed intraluminally and then connected to the fluid pressure source by forming an aperture in the wall of the aorta or other artery and connecting the fluid conducting tube from the fluid pressure source to the balloon or chamber via the aperture.

19. The heart assist device as claimed in claim 18, wherein the fluid conducting tube is a gas carrying tube which exits the body percutaneously.

20. The heart assist device as claimed in claim 19, wherein the gas carrying tube is connected to a port on the stent thoracoscopically.

21. The heart assist device as claimed in claim 2, wherein the stent is placed intraluminally and then connected to a fluid pressure source placed in the right chest through a sternotomy.

22. The heart assist device as claimed in claim 21, further including a liquid carrying tube is connected through an aortotomy to a port on the stent which is in communication with the interior of the balloon or chamber.

23. A method of assisting the functioning of a heart of a patient, the method including the steps of:
holding a heart assist device, that includes an intraluminal inflatable counter-pulsation balloon, or chamber, in place against a wall of an arterial vessel of the patient;
connecting the inflatable balloon or chamber to a fluid pressure source;
energizing the fluid pressure source to expand and contract the inflatable balloon or chamber in counter-pulsation with the heart of a patient into whom the balloon or chamber has been placed.

24. The method as claimed in claim 23, wherein the chamber or balloon is part of a stent comprising an expandable frame and the method includes the step of attaching the counter-pulsation balloon or chamber to the inside wall of the frame.

25. The method as claimed in claim 23, wherein the method also includes the step of packaging the stent, including the balloon or chamber, into a catheter delivery structure and introducing the structure into a suitable artery.

26. The method as claimed in claim 23, wherein the method also includes the step of placing the stent intraluminally and then connecting it to fluid pressure source by forming an aperture in the wall of the aorta or other artery and connecting a fluid conducting tube from the fluid pressure source to the balloon or chamber.

27. The method as claimed in claim 23 wherein, the method also includes the step of placing the stent intraluminally and then connecting it to a hydraulic driver placed in the right chest through a sternotomy.
